(19) United States
(12) Plant Patent Application Publication
(10) Pub. No.: US 2015/0334896 P1
(43) Pub. Date: Nov. 19, 2015

(54) Dianthus plant named 'KLEDP13155'

(50) ;

(71) Applicant: KLEMM+SOHN GMBH & CO. KG, Stuttgart (DE)

(72) Inventors: Nils Klemm, Stuttgart (DE); Martin Glawe, Stuttgart (DE)

(73) Assignee: KLEMM+SOHN GMBH & CO. KG, Stuttgart (DE)

(21) Appl. No.: 14/120,255

(22) Filed: May 13, 2014

(51) Int. Cl. A01H 5/00

(52) U.S. Cl. PLT/278

(57) ABSTRACT

A new Dianthus plant particularly distinguished by having double flowers with a stable, shiny red flower color, good branching, and strong vigor, is disclosed.

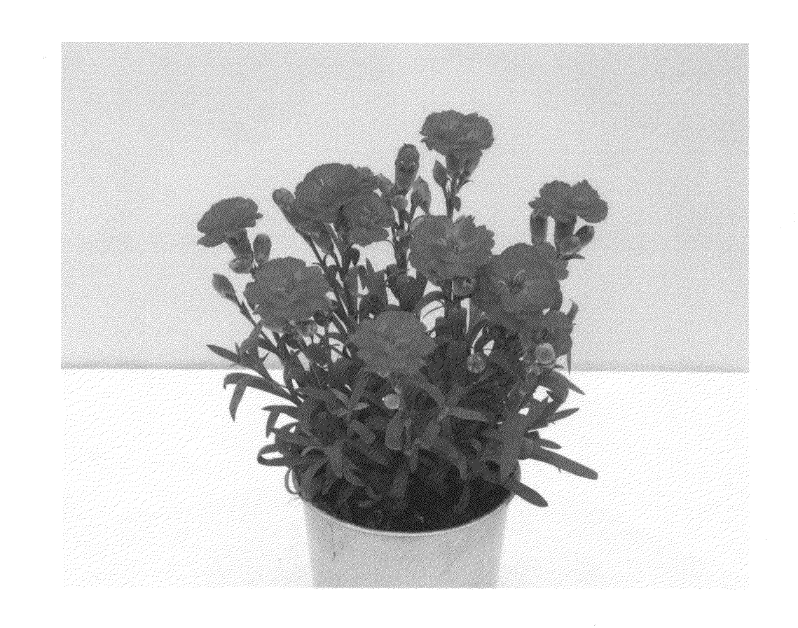

### GENUS AND SPECIES

Dianthus caryophyllus

### VARIETY DENOMINATION

‘KLEDP13155’

### BACKGROUND OF THE NEW PLANT

The present invention comprises a new and distinct variety of Dianthus, botanically known as Dianthus caryophyllus and hereinafter referred to by the variety name ‘KLEDP13155’. ‘KLEDP 13155’ is a selection that results from a cross pollination of the proprietary female dianthus line ‘DCP 2007 0056’ (unpatented) and the male dianthus line ‘Fire’ (patent status unknown).

In May of 2009 the proprietary female dianthus line ‘DCP 2007 0056’ and the male dianthus line ‘Fire’ were cross pollinated in Stuttgart, Germany and seeds were obtained. The seeds were sown and plants were grown for evaluation. A plant line was selected in June 2010 in Stuttgart, Germany, and named ‘KLEDP13155’. In June 2010, ‘KLEDP13155’ was first vegetatively propagated by vegetative cuttings. ‘KLEDP13155’ was found to reproduce true to type in successive generations of asexual propagation via vegetative cuttings in Stuttgart, Germany.

### SUMMARY

The following are the most outstanding and distinguishing characteristics of this new variety when grown under normal horticultural practices in Stuttgart, Germany.

1. Double flowers with a stable, shiny red flower color;

2. Good branching; and

3. Strong vigor.

### CROSS REFERENCE TO RELATED APPLICATIONS

Plant Breeder's Rights for ‘KLEDP13155’ were applied for in Canada on Jan. 20, 2014, application number 14-8175 and in Europe with the Community Plant Variety Office on Jun. 28, 2013, application number 2013/1747. ‘KLEDP13155’ has not been made publicly available or sold more than one year prior to the filing date of this application.

### DESCRIPTION OF THE PHOTOGRAPH

This new Dianthus plant is illustrated by the accompanying photograph which shows an overall view of the plant. The colors shown are as true as can be reasonably obtained by conventional photographic procedures. The plant is about 5 months-old grown in Stuttgart, Germany in May 2012.

### DESCRIPTION OF THE NEW VARIETY

The following detailed descriptions set forth the distinctive characteristics of ‘KLEDP13155’. The data which define these characteristics were collected from asexual reproductions carried out in Stuttgart, Germany. Data was collected on five month-old plants grown in a greenhouse in Stuttgart, Germany in February 2014. Plants were pinched one time with a growth regulator. Color references are to The R.H.S. Colour Chart of The Royal Horticultural Society of London (R.H.S.), 5^{th} edition (2007).
Classification:
   Family.—Caryophyllaceae.
   Species.—Dianthus caryophyllus.
   Common name.—Pot carnation.
   Variety.—‘KLEDP13155’.
Parentage:
   Female.—The proprietary dianthus ‘DCP 2007 0056’.
   Male.—‘Fire’.
Propagation:
   Type.—Vegetative cuttings.
   Time to produce a rooted young plant.—14 to 16 days.
   Root description.—White, fine.
   Rooting habit.—Lanceolate.
Plant description:
   Plant form.—Potted plant, upright.
   Plant height.—17.0 cm to 19.0 cm.
   Plant diameter or spread.—14.0 cm to 16.0 cm.
Lateral branches:
   Length.—9.0 cm.
   Diameter.—5.0 cm.
   Internode length.—1.5 cm to 2.0 cm.
   Node length.—0.4 cm.
   Node diameter.—0.3 cm.
   Texture.—Smooth.
   Color.—RHS 137A.
Foliage description:
   Arrangement.—Opposite.
   Length.—6.0 cm to 8.0 cm.
   Width.—0.7 cm.
   Shape.—Lanceolate.
   Apex.—Acute.
   Base.—Obtuse.
   Margin.—Entire.
   Texture.—Upper and lower surfaces: Smooth.
   Venation pattern.—Palmate.
   Color.—Immature leaves: Upper surface: RHS 144A Lower surface: RHS 146C Mature leaves: Upper surfaces: RHS 137A Lower surface: RHS 137C Venation: RHS 137A.
Flower description:
   Flower type and habit.—Double, round, small sprays.
   Fragrance.—Present.
   Natural flowering season.—Spring to fall.
   Flower longevity.—10 to 12 days.
   Flower buds.—Length: 2.0 cm to 2.4 cm Diameter: 1.0 cm Shape: Oval Color: RHS 143A, RHS 143C, RHS 137C and RHS 176A.
   Corolla.—Quantity of petals/arrangement: Greater than 20, arranged in several whorls Corolla height: 1.8 cm to 2.0 cm Petal length: 4.0 cm Petal width: 1.8 cm to 2.0 cm Petal shape: Spoon shaped Petal apex: Obtuse Petal base: Acute Petal margin: Crenate Petal texture: Upper and lower surfaces: Satiny Petal color: Immature: Upper surface: RHS 46B Lower surface: RHS 47B Mature: Upper surface: RHS 53C Lower surface: RHS 56A Calyx: Quantity of sepals/arrangement: 4 to 5, fused Calyx height: 2.0 cm Calyx diameter, at the apex: 1.0 cm to 1.2 cm Sepal texture: Upper and lower surfaces: Smooth Sepal color: Upper surface: RHS 144A Lower surface: RHS 145C.
Peduncles:
   Length.—16.0 cm to 18.0 cm.
   Diameter.—0.2 cm.
   Strength.—Moderate.
   Texture.—Smooth.
   Color.—RHS 137.
Reproductive organs:
   Stamens.—Quantity: 3 to 5 Filament length: 0.3 cm to 0.5 cm Filament color: RHS 158D Anther length: 0.2 cm Anther diameter: 0.1 cm Anther color: RHS 158A.
   Pistils.—Quantity: 1 Pistil length: 0.1 cm Style length: 2.5 cm Style color: RHS 155B Stigma shape: Filamentous Stigma color: RHS 60A Ovary size: 0.6 cm Ovary shape: Oval Ovary color: RHS 145A Fruits/seeds: None observed.
Disease and insect/pest resistance: No special observations were made

### COMPARISON WITH PARENTAL LINES AND KNOWN VARIETY

‘KLEDP13155’ is a distinct variety of dianthus. ‘KLEDP13155’ is distinguished from its parents as described in Table 1:

TABLE 1
Comparison with Parental Lines

| | Variety | Female parent | Male Parent |
|---|---|---|---|
| Trait | ‘KLEDP13155’ | ‘DCP 2007 0056’ | ‘Fire’ |
| Branching | Good | Less branching | Medium |
| Vigor | Strong | Medium | Medium |

‘KLEDP13155’ is most similar to the commercial dianthus plant named SuperTrouper ‘Benita’ (unpatented). Differences between the two varieties are described in Table 2:

TABLE 2
Comparison with Similar Variety

| | Variety | Commercial line |
|---|---|---|
| Trait | ‘KLEDP13155’ | SuperTrouper ‘Benita’ |
| Ray florets color | Shiny red, | Unstable |
| and stability | stable | color |

We claim:

1. A new and distinct variety of Dianthus plant named ‘KLEDP13155’ as shown and described herein.
